(19) United States
(12) Patent Application Publication
(10) Pub. No.: US 2012/0328574 A1
(43) Pub. Date: Dec. 27, 2012

(54) P38 INHIBITORS

(75) Inventors: Indravadan Ambalal Modi, Ahmedabad (IN); Prasanta Kumar Ghosh, Ahmedabad (IN); Devesh Bhardwaj, Ahmedabad (IN); Nirav Desai, Ahmedabad (IN); Bakulesh Mafatlal Khamar, Ahmedabad (IN)

(73) Assignee: Cadila Pharmaceuticals Ltd., Ahmedabad (IN)

(21) Appl. No.: 13/535,917

(22) Filed: Jun. 28, 2012

Related U.S. Application Data
(62) Division of application No. 12/532,100, filed on Sep. 18, 2009.
Application No. 12/532,100 is a division of application No. PCT/IB2008/000633, filed on Mar. 18, 2008.

(30) Foreign Application Priority Data
Mar. 20, 2007 (IN) 509/MUM/2007

(51) Int. Cl. A61K 35/74; A61P 37/02; A61P 19/08; A61P 9/10; A61P 37/08; A61P 25/28; A61P 35/00; A61P 29/00

(52) U.S. Cl. 424/93.4

(57) ABSTRACT

The invention relates to novel p38 MAPK inhibitor which involves Mycobacterium w and/or its constituents in pharmaceutically acceptable carriers and their uses. Mycobacterium w and/or its constituents when administered to mammal results in p38 inhibition The inhibition is found to last more than 28 days. It is also found to induce inhibition of TNF-α it suppresses cytokines in a pattern identical to Glucocorticoids. In transforms cells it also induces apoptosis. P38 mediated conditions include inflammation, cell differentiation, cell proliferation, cell inhibition, cell cycle regulation, anti-inflammatory reactions, immune modulation, vascularization, response to external stimuli and angiogenesis. The use of Mycobacterium w (Mw) and/or constituents of Mycobacterium w for inhibition of p38 protein kinase i.e. (i) to induce apoptosis in transformed cells (ii) for inhibition of TNF-α (iii) for inhibition of cytokines.

### CROSS REFERENCE TO PRIOR APPLICATIONS

This application is a divisional application and claims priority to and the benefit of co-pending U.S. Ser. No. 12/532,100, filed on Sep. 18, 2009, which is a U.S. national phase application under 35 U.S.C. §371 of International Patent Application No. PCT/IB2008/000633, filed Mar. 18, 2008, which claims the priority and the benefit of Indian Patent Application No. 509/MUM/2007, filed Mar. 20, 2.007, the entire contents of which are incorporated herein by reference,

### FIELD OF THE INVENTION

The current invention relates to novel p38 inhibitors, processes for the preparation thereof, the use thereof in treating p38 kinase mediated diseases and pharmaceutical compositions for use in such therapy.

### BACKGROUND OF THE INVENTION

Mitogen-activated protein kinases (MAPK) are a family of proline-directed serine/threonine kinases that activate their substrates by dual phosphorylation. The kinases are activated by a variety of signals including nutritional and osmotic stress, UV light, growth factors, endotoxin and inflammatory cytokines.

One particularly interesting MAPK is p38, also known as cytokine suppressive anti-inflammatory drug binding protein (CSBP). The p38 kinases are responsible for phosphorylating and activating transcription factors as well as other kinases. They are activated by physical, chemical, and radiation stresses like osmotic, anisomysin, UV etc. They are also activated by pro-inflammatory cytokines like IL-1 and TNF and bacterial lipopolysaccharide. More importantly, the products of the p38 phosphorylation activation have been shown to mediate the production of inflammatory cytokines, including TNF, IL-1, IL-6 and cyclooxygenase-2. Each of these cytokines has been implicated in numerous disease states and conditions.

P38-mediated conditions include any disease or deleterious condition in which upregulated p38 plays a role in pathogenesis of that condition and/or inhibition of p38 is useful in management of the same p38-mediated conditions include inflammatory diseases, autoimmune diseases, destructive bone disorders, proliferative disorders including tumor progression, infectious diseases, neurodegenerative diseases, allergies, reperfusion/ischemia in stroke, heart attacks, angiogenic disorders, organ hypoxia, vascular hyperplasia cancer cachexia, cardiac hypertrophy, thrombin-induced platelet aggregation, and conditions associated with prostaglandin endoperoxidase synthase-2. p38 has been implicated in cancer, immunodeficiency disorders, cell death and osteoporosis.

Inhibition of p38 kinase leads to a blockade on the production of both IL-1 and TNF. IL-1 & TNF stimulate the production of other pro-inflammatory cytokines such as IL-6, and IL-8, which have been implicated in acute and chronic inflammatory diseases and in post menopausal osteoporosis [R. B. Kimble et al., Endocrinol., 136, pp. 3054-61, (1995)]. The diseases characterized with abnormal regulation of these cytokines are amenable to treatment with p38 inhibitor.

IL-1-mediated disease or condition includes rheumatoid arthritis, osteoarthritis, stroke, endotoxemia and/or toxic shock syndrome, inflammatory reaction induced by endotoxin, inflammatory bowel disease, tuberculosis, atherosclerosis, muscle degeneration, cachexia, psoriatic arthritis, Reiter's syndrome, gout, traumatic arthritis, rubella arthritis, acute synovitis, diabetes, pancreatic beta-cell disease and Alzheimer's disease.

TNF-α levels can be altered by a variety of pharmaceutical compositions that are currently being used in mammals. Such compositions have TNF-α antagonist activity, and include Infliximab, Adalulimb, Etarncept, Thalidomide, etc. They are used in management of rheumatoid arthritis, Crohn's disease, Ankylosing spondylitis, ulcerative colitis, apthous ulcer, systemic lupus erythematous, myeloma, uveitis, etc.

Glucocorticoids are known anti-inflammatory compounds. Commonly used glucocorticoids include hydrocortisone, prednisolene, betamethasone, dexamethasone, triaminolone, methyl prednisolene, prednisone. Glucocorticoids suppress cytokines like IL-1, IL-2, IL-3, IL-4, IL-5, IL-6, IL-8, IL-11, IL-12, TNF-α COX-2, IL-1, IL-2, IL-6, IL-8, IL-12, TNF-α, which are known as proinflammatory cytokines, while IL-4, IL-5, etc. are known as anti-inflammatory cytokines. Glucocorticoids are used in management of wide range of diseases which include rheumatoid arthritis, rheumatoid spondylitis, asthma, atopic dermatitis, drug hypersensitivity reactions, perennial or seasonal allergic rhinitis, serum sickness, bullous dermatitis herpetiformis, exfoliative erythroderma, mycosis fungoids, pemphigus, severe erythema multiforme(Stevenes), ulcerative colitis, idiopathic thrombocytopenic purpura, pure red cell aplasia, temporal arteritis, uveitis, proteinuria in idiopathic nephritis, idiopathic eosinophilic pneumonias, symptomatic sarcoidosis, acute gouty arthritis, ankylosing spondylitis, dermatomyositis, polymyositis, systemic lupus, refractory multiple myeloma, myelodysplastic syndromes, severe COPD, chronic granulomatous disease, angiogenesis, sarcoidosis.

Transformed cells are the cells, which grow into continuous culture without mitogen stimuli. Eukaryotic cells are non transformed cells and do not grow in continuous culture. By transformation eukaryotic cells get converted from quiescent/stationary phase to unregulated, growth and can be maintained in continuous culture. The p38 inhibitors are known to inhibit continuous growth of these transformed cells and trigger apoptosis.

Following patents, patent applications describe p38 inhibitors and uses thereof

| | |
|---|---|
| U.S. Pat. No. 7,186,737B2 Inhibitors of p38 | U.S. Pat. No. 7,169,779B2 Inhibitors of p38. |
| U.S. Pat. No. 6,635,644B2 Inhibitors of p38 | U.S. Pat. No. 6,608,060B1 Inhibitors of p38 |
| U.S. Pat. No. 6,632,945B2 Inhibitors of P38 | U.S. Pat. No. 6,528,508B2 Inhibitors of p38 |
| U.S. Pat. No. 6,509,363B2 Heterocyclic inhibitors of p38 | U.S. Pat. No. 6,147,080A Inhibitors of p38 |
| U.S. Pat. No. 6,800,626B2 Inhibitors of p38 | U.S. Pat. No. 6,093,742A Inhibitors of p38 |
| U.S. Pat. No. 6,949,560B2 Imidazo-substituted compounds | W02000017175A1 Inhibitors of p38 |
| as p38 kinase inhibitors | W02000017204A1 Inhibitors of p38 |
| W01996021654A1 Novel Compounds | W01999058502A1 Heterocyclic |
| W01999000357A1 Inhibitors of p38 | inhibitors of p38 |
| W01999064400A1 Inhibitors of p38 | |

U.S. Pat. No. 6,162,613A Methods for designing inhibitors of serine/threonine-kinases and tyrosine kinases
U.S. Pat. No. 715,101 OB2 Methods for assembling a stack package for high density integrated circuits
U.S. Pat. No. 6,852,740B2 Pyrazole derivatives as p38 kinase inhibitors
U.S. Pat. No. 6,982,270B1 3,4-dihydro-(1h)quinazolin-2-one compounds as csbp/p38 kinase inhibitors
U.S. Pat. No. 6,630,485B2 p38 map kinase inhibitor
U.S. Pat. No. 7,189,400B2 Methods of treatment with antagonists of mu -1
U.S. Pat. No. 7,115,557B2 Use of certain drugs for treating nerve root injury
U.S. Pat. No. 7,078,431B2 1,3-bis-(substituted-phenyl)-2-propen-1-ones and their use to treat vcam-1 mediated disorders
U.S. Pat. No. 6,759,410B2 3,4-dihydro-(1h)-quinazolin-2ones and their use as csbp/p38 kinase inhibitors
U.S. Pat. No. 6,696,471B2 Aminopyrrole compounds
U.S. Pat. No. 6,696,443B2 Piperidine/piperazine-type inhibitors of p38 kinase
U.S. Pat. No. 6,649,637B2 Inhibition of intracellular replication by pyridinylimidazoles
U.S. Pat. No. 6,638,765B1 Platform for the differentiation of cells U.S. Pat. No. 6,509,361B1 1,5-diaryl substituted pyrazoles as p38 kinase inhibitors
U.S. Pat. No. 6,479,507B2 p38 map kinase inhibitors
U.S. Pat. No. 6,444,696B1 pyrazole derivatives p38 map kinase inhibitors
U.S. Pat. No. 6,410,540B1 Inhibitors of P38 alpha kinase
U.S. Pat. No. 6,376,527B1 Pyrazole derivatives P38 Map kinase inhibitors
U.S. Pat. No. 6,316,466B1 Pyrazole derivatives P38 Map kinase inhibitors
U.S. Pat. No. 6,316,464B1 P38 Map Kinase Inhibitors
U.S. Pat. No. 6,096,711A HSP 72 Induction And Applications
U.S. Pat. No. 6,414,150B1 & describes inhibition of angiogenesis by suppression of TNF-alpha
U.S. Pat. No. 6,335,336B1 is useful in inhibition or prevention of metastasis.
U.S. Pat. No. 6,994,981B2 describe modulators of para apoptosis and related methods. Several other prior art patents are also based on MAPK inhibitors are EP1208748A1, WO2004089929, WO2006117567.
U.S. Pat. No. 6,852,740B2 describes pyrazole derivatives as p38 kinase inhibitors. WO95/31451 describes pyrazole compositions that inhibit MAPKs, and, in particular, p38. The efficacy of these inhibitors in vivo is still being investigated.

Other p38 inhibitors have been produced, including those described in WO98/27098,WO99/00357, WO99/10291, WO99/58502, WO99/64400, WO00/17175 and WO00/17204, In addition, WO97/24328, WO98/34920, WO98/35958 and U.S. Pat. No. 5,145,857A disclose amino substituted heterocycles having therapeutic uses.

Accordingly, there is a need to develop inhibitors of p38 that are useful in treating various conditions associated with p38 mediated activity.

### SUMMARY OF THE INVENTION

One embodiment of the present invention is to provide Mycobacterium w (Mw) cells and/or its constituents for p38 kinase inhibition.

It is another embodiment of the invention to provide methods for treatment or prevention of a p38-mediated condition.

It is yet another embodiment of the invention to provide a method for the treatment of condition or disease state mediated by p38 kinase activity, or mediated by cytokines produced by the activity of p38 kinase, which comprises administering to a subject (e.g. mammals) a therapeutically effective amount of Mycobacterium w and/or constituents thereof.

It is yet another embodiment of the invention to provide use of Mycobacterium w and/or constituents thereof, for the preparation of a medicament for the treatment of a condition or disease state mediated by p38 kinase activity or medicated by cytokines produced by p38 kinase activity.

### BRIEF DESCRIPTION OF THE DRAWING

FIG. 1 depicts in vitro studies demonstrating Mycobacterium w reduces P38 level.

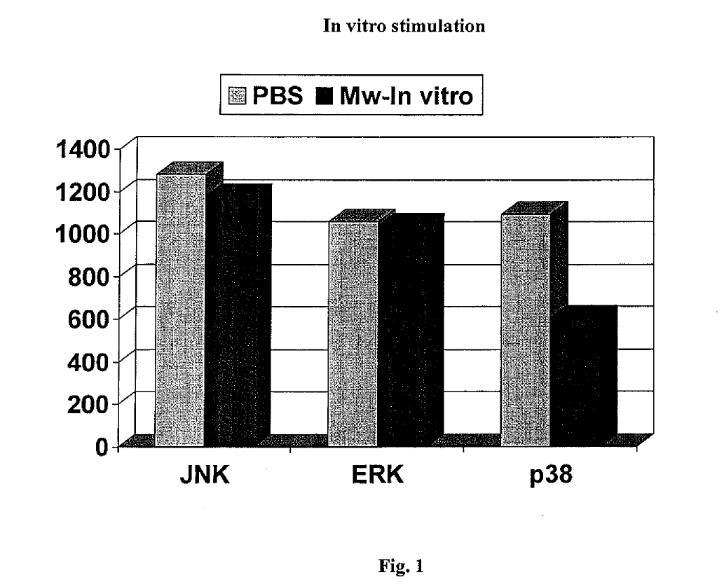

FIG. 2 depicts in vivo studies demonstrating Mycobacterium w reduces P38 level.

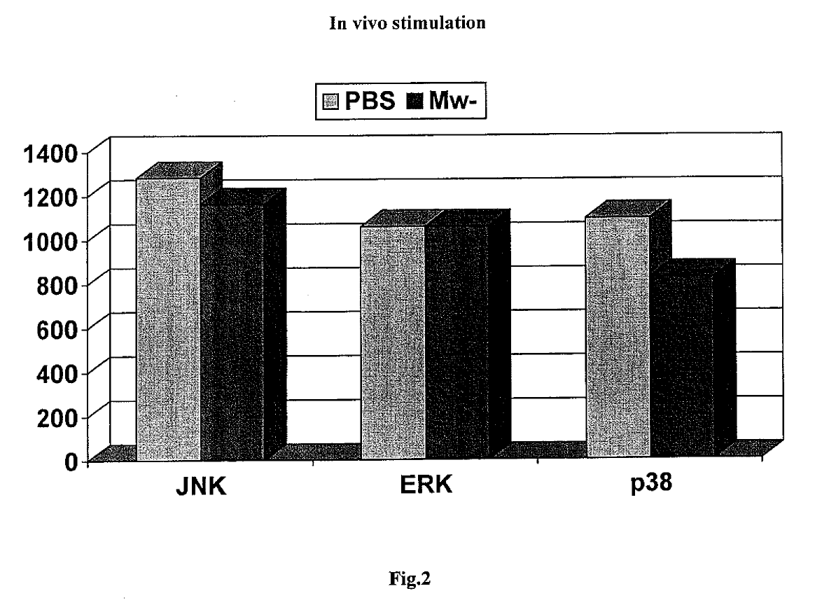

### DETAILED DESCRIPTION OF THE INVENTION

In one embodiment, the present invention relates to the use of Mycobacterium w (Mw) cells and/or its constituents fur inhibition of p38 protein kinase.

Another embodiment of the present invention includes the use of Mw cells and/or its constituents for inhibition of cytokine production.

Another embodiment of the present invention encompasses compositions comprising Mw cells and/or its constituents are inhibitors of serine/threonine kinase p38 and cytokine production.

In accordance with the invention Mycobacterium w (Mw) cells and/or its constituents may be useful in treating p38 mediated disorders.

The invention comprises compositions having therapeutically effective amount of Mw cells and/or its constituents for the treatment of p38 kinase mediated disorder, TNF mediated disorder, inflammation and/or arthritis.

The present invention provides a method of treating a cytokine-mediated disease which comprises administering an effective cytokine interfering amount of compositions containing Mw and/or its constituents. The use includes but is not limited to rheumatoid arthritis, rheumatoid spondylitis, asthma, atopic dermatitis, drug hypersensitivity reactions, perennial or seasonal allergic rhinitis, serum sickness, bullous dermatitis herpetiformis, exfoliative erythroderma, mycosis fungoids, pemphigus, severe erythema multiforme (Stevenes), ulcerative colitis, idiopathic thrombocytopenic purpura, pure red cell aplasia, temporal arthritis, uvetitis, proteinuria in idiopathic nephritis, idiopathic eosinophilic pneumonias, symptomatic sarcoidosis, acute gouty arthritis, ankylosing spondylitis, dermatomyositis, polymyositis, systemic lupus, refractory multiple myeloma, myelodysplastic syndromes, severe CoPD, Chronic granulomatous disease, angiogenesis, sarcoidosis.

Mw cells are useful for the treatment of p38 kinase mediated disorder including inflammatory diseases, autoimmune diseases, destructive bone disorders, proliferative disorders including tumor progression, infectious diseases, neurodegenerative diseases, allergies, reperfusion, ischemia in stroke, heart attacks, angiogenic disorders, organ hypoxia, vascular hyperplasia cancer cachexia, cardiac hypertrophy, thrombin-induced. platelet aggregation, conditions associated with prostaglandin endoperoxidase synthase-2, cancer, immunodeficiency disorders, cell death, osteoporosis.

Mw cells may be used for the treatment of TNF-α mediated disease or condition including rheumatoid arthritis, crohn's disease, ankylosing spondylitis, ulcerative colitis, apthous systemic lupus erythematous, myeloma uveitis.

Mw cells and/or its constituents involved in the said invention-may also be used in co-therapies, partially or completely, in place of other conventional anti-inflammatories, such as together with steroids, Dexamethasone, cyclooxygenase-2 inhibitors, NSAIDs, DMARDS, immunosuppressive agents, 5-lipoxygenase inhibitors, LTb4 antagonists and LTA, hydrolase inhibitors.

In accordance with the invention, Mw cells may be used to inhibit p38 mediated conditions, in which Mw cells are prepared by the process comprises the following steps;
a. Culturing of Mycobacterium w (Mw),
b. Harvesting and concentrating,
c. Washing the cells,
d. Adding pharmaceutically acceptable carrier,
e. Adding preservative,
f. Terminal sterilization,
g. Quality control,
h. Preparing constituents of Mw.
   The process is further described in detail is as following:
A. Culturing of Mw:

i. Culturing Mw on solid medium like L J medium or liquid medium like middle brook medium or Sauton's liquid medium. For better yield middle brook medium is enriched. It can be preferably enriched by addition of glucose, bactotryptone, and BSA. They are used in ratio of 20:30:2 preferably. The enrichment medium is added to middle brook medium. It is done preferably in ratio of 15:1 to 25:1 more preferably in a ratio of 20:1. Preparing the culture medium at 37+/−05° C. temperature and at pH 6.7 to 6.8 initially.

ii. Bioreactor operation
a) Preparation of vessel: Cleaning the inner contact parts of the vessel (Joints, mechanical seals, o-ring/gasket grooves, etc.) to avoid contamination. Filling the vessel with 0.1 N NaOH and leave for 24 hrs to remove pyrogenic material and other contaminants. Cleaning the vessel with acidified water and then with water. Rinsing the vessel with distilled water.
b) Sterilization of bioreactor: Sterilizing the bioreactor containing 9 L, distilled water with steam. Further sterilizing the bioreactor with Middlebrook medium. Bottles, inlet/outlet air filters etc. are autoclaved (twice) at 121° C. for 15 minutes. Drying the vessel in oven at 50° C. before use.

B. Harvesting and concentrating: Harvesting the cells under aseptic condition at the end of the 6th day of culturing. Concentrating the cells (pelletization) by centrifugation.

C. Washing cells: Washing the pellet with normal saline, preferably with isotonic fluid.

D. Addition of pharmaceutically acceptable carrier: Adding pyrogen free normal saline to pellet. Any other pyrogen free isotonic fluid can be sued as a pharmaceutical carrier. The carrier is added in amount so as to get desired concentration of active in final form.

E. Addition of preservative: Adding preservative to keep the cell/pellets free from contamination. Preferably thiomerosal is used having concentration of 0.01% w/v.

F. Terminal Sterilization: Sterilizing the cell/pellet by various physical methods like application of heat or ionizing radiation or sterile filtration. Heat can be in the form of dry heat or moist heat. It can also be in the form of boiling or pasteurization. Ionizing radiation can be Ultraviolet or gamma rays or microwave or any other from.

G. Quality Control: The cell/pellet passed through number of process to check its quality.

i. Evaluating purity and sterility of the cell/pellet.

ii. Checking the organisms for acid fastness after gram staining.

iii. Performing Inactivation test by culturing the product on L J medium to find out any living organism.

iv. Checking pathogenicity and/or contamination of the cell/pellet. The cultured organisms are injected to Balb/c mice. All the mice gained weight and found healthy. Three is no macroscopic or microscopic lesions seen in liver, lung spleen or any other organs of the mice.

v. Biochemical Test: The cell/pellet containing organism is subjected to following biochemical tests:
Urease—Tween 80 hydrolysis
Niacin test—Nitrate reduction test

The organism gives negative results when tested with urease, tween 80 hdrolysis and niacin. It gives positive result with nitrate reduction test.

H. Preparation of Mycobacterium w constituents: Mw constituents can be prepared by the following methods:
i. Cell disruption
ii. Solvent extraction
iii. Enzymatic extraction.

The cell disruption is done by sonication or using of high pressure fractionometer or applying osmotic pressure.

The solvent extraction is done with any organic solvent like chloroform, ethanol, methanol, acetone, phenol, isopropyl alcohol, acetic acid, urea, hexane etc.

The enzymatic extraction is done with proteolytic enzymes which can digest cell wall/membranes. Liticase and pronase are the preferred enzymes. Mw cell constituents can be used in place of Mw. Addition of Mw cell constituents results in improved efficacy of the product. Cell/pellet containing Mw so prepared is further evaluated for its p38 inhibiting activity,

In accordance with this invention, Mw cell prepared by the aforementioned process is used in the preparation of pharmaceutical compositions.

A. Each dose of 0.1 ml of therapeutic agent contains:

| | |
|---|---|
| Mycobacterium w., (heat killed) | 0.50 × 10⁹ |
| Sodium Chloride 1.P. | 0.90% w/v |
| Tween 80 | 0.1% w/v |
| Thiomerosal 1.P. | 0.01% w/v (As a Preservative) |
| Water for injection 1.P. | q.s. to 0.1 ml |

Each dose of 0.1 ml of therapeutic agent contains:

| | |
|---|---|
| Mycobacterium w., (heat killed) | 0.50 × 10⁹ |
| Sodium Chloride 1.P. | 0.90% w/v |
| Triton x 100 | 0.1% w/v |
| Thiomerosal I.P. | 0.01% w/v (As a Preservative) |
| Water for injection I.P. | q.s. to 0.1 ml |

C. Each dose of 0.1 ml of therapeutic agent contains:

| | |
|---|---|
| Mycobacterium w., (heat killed) | 0.50 × 10⁹ |
| Sodium Chloride 1.P | 0.90% w/v |
| Thiomerosal I.P. | 0.01% w/v (As a Preservative) |
| Water for injection 1.P. | q.s. to 0.1 ml |

D. Each dose of 0.1 ml of therapeutic agent contains Extract of Mycobacterium w after sonication from 1×10¹⁰ Mycobacterium w

| | |
|---|---|
| Sodium Chloride 1.P. | 0.90% w/v |
| Thiomerosal 1.P. | 0.01% w/v (As a Preservative) |
| Water for injection 1.P. | q.s. to 0.1 ml |

E. Each dose of 0.1 ml of therapeutic agent contains Methanol Extract of 1×10¹⁰ Mycobacterium w

| | |
|---|---|
| Sodium Chloride I.P. | 0.90% w/v |
| Thiomerosal 1.P. | 0.01% w/v (As a Preservative) |
| Water for injection I.P. | q.s. to 0.1 ml |

F. Each dose of 0.1 ml of therapeutic agent contains: Chloroform Extract of 1×10¹⁰ Mycobacterium w

| | |
|---|---|
| Sodium Chloride I.P. | 0.90% w/v |
| Thiomerosal I.P. | 0.01% w/v (As a Preservative) |
| Water for injection 1.P. | q.s. to 0.1 ml |

G. Each dose of 0.1 ml of therapeutic agent contains Acetone Extract of 1×10¹⁰ Mycobacterium w

| | |
|---|---|
| Sodium Chloride I.P. | 0.90% w/v |
| Thiomerosal I.P. | 0.01% w/v (As a Preservative) |
| Water for injection I.P. | q.s. to 0.1 ml |

H. Each dose of 0.1 ml of therapeutic agent contains Ethanol Extract of 1×10¹⁰ Mycobacterium

| | |
|---|---|
| Sodium Chloride I.P. | 0.90% w/v |
| Thiomerosal I.P. | 0.01% w/v (As a Preservative) |
| Water for injection I.P. | q.s. to 0.1 ml |

I. Each dose of 0.1 ml of herapeutic agent contains Liticase Extract of 1×10¹⁰ Mycobacterium w

| | |
|---|---|
| Sodium Chloride I.P. | 0.90% w/v |
| Thiomerosal I.P. | 0.01% w/v (As a Preservative) |
| Water for injection I.P. | q.s. to 0.1 ml |

J. Each dose of 0.1 mi of therapeutic agent contains Mycobacterium w (heat killed) 0.5×10⁷ Extract of Mycobacterium w obtained 1×10³ Mycobacterium w by disruption, solvent extraction or enzymatic extraction.

| | |
|---|---|
| Sodium Chloride I.P. | 0.90% w/v |
| Thiomerosal I.P. | 0.01% w/v (As a Preservative) |
| Water for injection I.P. | q.s. to 0.1 ml |

The amount of Mw cell that may be combined with the carrier materials to produce a single dosage form will vary depending upon the host treated and the particular mode of administration.

The route of administration can be injection intradermal, intra venous, intra vesicle, intra peritoneal, intra articular, intra cerebral, intramuscular, sub cutaneous or any other route known in art for the particular treatment. For transdermal administration, the pharmaceutical composition may be given in the form of a transdermal patch, such as a transdermal iontophoretic patch.

The pharmaceutical compositions so manufactured are surprisingly found to have following properties. They include p38 inhibitors, TNF-α inhibitor, suppression of cytokines and death of transformed cells.

The concentration at which death of transformed cell take place is safe for normal cells like splenocytes, PBMC, bone marrow cell, fibroblasts, macro phages, etc.

The invention is further illustrated with the following examples which do not limit the scope of the invention.

### EXAMPLE 1

### In Vivo p38 Inhibition by Mw by Intra Dermal Route

Naive Balb/C mice were divided in two randomized groups. All mice received intradermal injections. The first group received 100 mcL of PBS, second group received 100 mcL of Mw (10̂8 cells). On eighth day mice were sacrificed and spleens were isolated from all animals. The Splenocytes were isolated from each group and cultured in RPMI 1640 media with 10% Fetal Bovine Serum (FBS) and 1% antibiotics in inicrotitre plate. After 48 hrs of culture the cells were harvested and the cell signaling assays were performed as per manufacturers instructions, using the commercial kits (Cat no # DYC869-5) from R&D Systems.

The result depicted in Table 1 show significant inhibition of p38 MAPK following intradermal administration of pharmaceutical composition of present invention.

TABLE I
p38 MAPK inhibition in vivo by Mycobacterium w in normal cells

| | Route of immunization & | In vivo % |
|---|---|---|
| Cell type | dose | Inhibition |
| Normal cells (Splenocytes 10\hat{ }6) | Intra venous Mw 10\hat{ }9 cells | 20% |
| Normal cells (Splenocytes 10\hat{ }6) | Intra dermal Mw 10\hat{ }8 cells | 19% |

### EXAMPLE 2

### In Vivo p38 Inhibition by Mw with Intra Venous Route

Naive Balb/C mice were divided in two randomized groups. All mice received intravenous injection of a PBS (Placebo) of Mw. The first group received, 100 mcL of PBS, second group received 100 mcL of Mw (10̂8 cells). On eighth day mice were sacrificed and spleens were isolated from all animals. The Splenocytes were isolated from each group and cultured in RPMI 1640 media with 10% Fetal Bovine Serum (FBS) and 1% antibiotics in microtitre plate. After 48 hrs of culture the cells were harvested and the cell signaling assays were performed as per manufacturers instructions, using the commercial kits (Cat no #DYC869-5) from R&D Systems.

The result depicted in Table 1 show significant inhibition of p38 following administration of Mw by intra venous route.

### EXAMPLE 3

### In Vitro Inhibition of p38 by Mw

Naive Balb/C mice were sacrificed and spleens were isolated. The Splenocytes were isolated and cultured in RPMI 1640 media with 10% FBS and 1% antibiotics in microtitre plate. The number of wells were divided into two sets one was stimulated with 100 mcL of Mw (10̂8 cells) and second set was stimulated with 100 mcL placebo (PBS). After 48 hrs of incubation the cells were harvested and the cell signaling assays were performed as per manufacturers instructions, using the commercial kits (Cat no #DYC869-5) from R & D Systems.

The result depicted in Table 2 shows down regulation of p38 MAPK significantly when in vitro incubation of mice splenocytes with Mw.

TABLE 2
Inhibition of p38 MAPK by in vitro stimulation with
Mycobacterium w in normal and transformed cells

| | In vitro % |
|---|---|
| Cell type | inhibition |
| Normal cells (Splenocytes 10\hat{ }6) | 47% |
| Transformed cells (Mia-pa-ca-2 10\hat{ }5) | 39% |
| Transformed cells (NFS 60 10\hat{ }5 cells) | 18% |

### EXAMPLE 4

### p38 Inhibition in NFS-60 Cells by Mw

NFS 60 cells were cultured in Dulbecco's Minimal Eagle's Media (DMEM) with 10% FBS, 1% antibiotics and IL-3 10 nG/mL. The cells were plated in microtiter wells at concentration of 1×10̂5 cells. The numbers of wells were divided into two sets. Set one was stimulated with PBS as control and set two with 4×10̂6 Mw cells. At 24 hrs of culture the cells were harvested and the cell signaling assays were performed as per manufacturers instructions, using the commercial kits (Cat no #DYC869-5) from R&D Systems.

The result depicted in Table 2 shows down regulated level of p38 levels in Mw stimulated cells compared to control (non stimulated cells) at 24th hrs. At all the concentration above 4×10116 Mw cells, cell death was observed at 48 hrs, Cell death seen was due to apoptosis.

### EXAMPLE 5

### p38 Inhibition in Mia-pa-ca 2 Cells by Mw

Mai-pa-ca 2 cells (pancreatic cancer cell line) were obtained from ATCC and were cultured in DMEM media with 10% FBS, 1% antibiotics. The cells were plated in microtiter wells at concentration of 1×10115 cells. The numbers of wells were divided into two sets. Set one was stimulated with PBS as control and set two with 2×10116 Mw cells. At 48 hrs of culture the cells were harvested and the cell signaling assays were performed as per manufacturers instructions, using the commercial kits (Cat no #DYC869-5) from R & D Systems.

The result depicted in Table 2 shows down regulated level of p38 levels in Mw stimulated cells compared to control (non stimulated cells) at 48th hrs. At a concentration of Mw above 107 Mia-pa-ca 2 cells found to undergo apoptotic cell death.

### EXAMPLE 6

### Inhibition of p38 MAPK with Single Injection Compared to Seven Injections of Mycobacterium w Administered Intradermally

Naive Balb/C mice were divided in three randomized groups. All mice received drugs Intradermally. The first group received 100 mcL of PBS, second group received 100 mcL of Mw (10̂8 cells) once only, while third group was immunized with 100 mcL of Mw (1018 cells) every day for seven days. On eighth day after first immunization, mice were sacrificed and spleens were isolated for all three groups. The Splenocytes were isolated from each group and cultured in RPMI 1640 media with 10% FBS and 1% antibiotics in microtitre plate.

After 48 hrs of culture the cells were harvested and the cell signaling assays were performed as per manufacturers instructions, using the commercial kits (Cat no #DYC869-5) from R&D Systems.

The results shows administration of single injection of Mw inhibits p38 MAPK by 20%, while seven injections inhibit p38 levels by 25% compared to control.

### EXAMPLE 7

### Duration of p38 Inhibition by Mw

Naive Balb/C mice were randomized in six groups and were administered intravenously 1 mL of PBS in group one while group two to six received 1 mL Mw (10̂9 cells). The group 1 and 2 were sacrificed on day 1, while group three on 7 day, group four on 14 day, group five on 21 day, group six on 28 day and spleens were isolated. The Splenocyte were isolated and cultured in RPMI 1640 media with 10% FBS and 1% antibiotics in microtitre plate. After 48 hrs cells were harvested and the MAPK ELISA were performed as per manufacturers instructions, using the commercial kits (Cat no #DYC869-5) from R & D Systems.

The result depicted in Table 3 shows p38 level down regulated when immunization with Mw cells from 24 hrs to 28th day (17.4% and 17.3%). The maximum inhibition of p38 occurs on 14th day (25.1%). p38 level remains inhibited for the entire period of study (i.e. 28 days).

TABLE 3
Inhibition of p38 MAPK by in vivo stimulation with
Mycobacterium w intravenous immunization in mice

| Normal cells (Splenocytes 10116) | % inhibition |
|---|---|
| O hrs after immunisation | — |
| 1 day after immunisation | 17.4 |
| 7 days after immunisation | 20.2 |
| 14 days after immunisation | 25.1 |
| 21 days after immunisation | 14.1 |
| 28 days after immunisation | 17.3 |

### EXAMPLE 8

### Inhibition of p38 MAPK by Mw: Dose Dependent Effect

Naive Balb/C mice were sacrificed and spleens were isolated. The Splenocytes were isolated and cultured in RPMI 1640 media with 10% FBS and 1% antibiotics in microtitre 25 plates. The number of wells were divided into three sets one was stimulated with 100 mcL placebo (PBS). The second set was stimulated with 100 mcL of Mw (10̂8 cells). The third set was stimulated with 100 mcL of Mw (10̂6 cells). After 48 hrs of incubation the cells were harvested and the cell signaling assays were performed as per manufacturers instructions, using the commercial kits (Cat no #DYC869-5) from R&D Systems.

The result shows that in vitro incubation of splenocytes with Mw 10̂8 cells down regulate p38 MAPK by 46% while 10̂6 Mw cells have 5% inhibitory effect.

### EXAMPLE 9

### p38 MAPK Inhibition in NFS-60 Cells by Mw in Dose Dependent Manner

NFS 60 cells were cultured in DMEM media with 10% FBS, 1% antibiotics and IL310 nG/mL. The cells were plated in microtiter wells at concentration of lx 10̂5 cells. The numbers of wells were divided in to five sets. Set one was stimulated with PBS as control, set two with 6×10̂7 Mw cells, set three with 333 10̂7 Mw cells, set four with 7×10̂6 Mw cells, set five with 4×10/16 Mw cells. At 24 hrs of culture the cells were harvested and the cell signaling assays were performed as per manufacturers instructions, using the commercial kits (Cat no #DYC869-5) from R & D Systems.

The result depicted in Table 4 shows, alteration in p38 levels in Mw compared to control at 24th hrs it is down regulated. The dose dependency is in inverse relation to the Mw concentration. The maximum inhibition was observed with 4×10̂6 Mw cells. At all the concentration above 4×10̂6 Mw cells use for the stimulation. NTS 60 cells do not live for more than 48 hrs. The cells are found to undergo cell death by apoptosis.

TABLE 4
Inhibition of p38 MAPK in transformed cells

| Cell type | Group | In vitro % inhibition |
|---|---|---|
| Transformed cells | Control (PBS) | |
| (NFS 60 10\hat{ }5 cells) | Mw 6 × 10\hat{ }7 | 12% |
| | Mw 3 × 10\hat{ }7 | 13% |
| | Mw 4 × 10\hat{ }6 | 19% |

### EXAMPLE 10

### TNF-α Inhibition by Mw

Naive Balb/C mice were randomized in two groups. Mice from groups 1 and 2 were sacrificed and spleens were isolated. The Splenocytes were isolated and cultured in RPMI 1640 media with 10% FBS and 1% antibiotics in microtitre plate. Group 1 was incubated with PBS while group 2 was incubated with 10̂8 Mw cells. After 48 hrs the cell supernatant was separated and the levels of TNF-α were checked using commercial kit from R & D systems (Cat #MTA00).

The result depicted in Table 5 shows incubated of TNF-α in group stimulated with Mw. Surprisingly it is observed that TNF-α inhibition is around 74% while p38 inhibition is 5 only around 47%

TABLE 5
Inhibition of TNF-α production by Mycobacterium w
TNF-α Inhibition by Mw

| | | In vitro inhibition | |
|---|---|---|---|
| Cell type | Splenocytes 10⁶ cells | Amount (nG/mL) | % Inhibition |
| TNF-α | Control(PBS) | 193.5 | |
| | Mw 10⁸ | 49.5 | 74% |

Thus TNR-mediated disease or condition that can be treated according to present invention, but are not limited to includes, rheumatoid arthritis, crohn's disease, ankylosingspondylitis, ulcerative colitis, apthous ulcer, systemic lupus erythematous, myeloma uveitis and said management of mediated disorders comprises treating a subject having or susceptible to such disorder with a therapeutically-effective amount of Mw and/or Mw constituents.

### EXAMPLE 11

### Cytokine Suppression by Mw

Naive Balb/C mice were randomized in two groups. Mice from groups 1 and 2 were sacrificed and spleens were isolated. The Splenocytes were isolated and cultured in RPMI incubated 1640 media with 10% FBS and 1% antibiotics in microtitre plate. Group 1 was incubated with PBS while group 2 was incubated with 10̂8 Mw cells. After 48 hrs the cell supernatant was separated and the levels of cytokines were checked using commercial kit from R & D systems (Cat #M2000, Cat #M4000B, Cat #M1240).

The result depicted in Table 6 shows inhibited of cytokine 1L-2, IL-4, IL-5 and IL-12 p40 in group two incubated with Mw.

Surprisingly it is observed that all types of cytokines are inhibited. The effect is significantly more than amount of p38 inhibition (64% for IL-12p40 to 95% for IL-4 with a p38 inhibitory activity of around 47%).

TABLE 6
Inhibition of cytokine production by Mycobacterium w

| | | In vitro inhibition | |
|---|---|---|---|
| Cell type | Splenocyte 10⁶ cells | Amount (nG/mL) | /o Inhibition |
| IL-2 | Control(PBS) | 176 | 79% |
| | Mw 10⁸ | 37.67 | |
| | Control(PBS) | 292.5 | 95% |
| IL-4 | Mw 10⁸ | 13.92 | |
| | Control(PBS) | 61.67 | 85% |
| IL-5 | Mw 10⁸ | 9.17 | |
| | Control(PBS) | 38.52 | 64% |
| IL-12p40 | Mw 10⁸ | 13.70 | |

### EXAMPLE 12

### Comparison with Dexamethasone for Cytokine Suppression

Naive Balb/C mice were sacrificed and spleens were isolated for all five groups. The splenocytes were isolated from each group and cultured in RPMI 1640 media with 10% antibiotics in microtitre plate. The cells were plated in micro titer plate. The wells were divided into five sets. Set one was the control, set two was stimulated with Mw, set three with 10 mM of Dexamethasone set four with 10 mcM (micro mole) of Dexamethasone and set five with 1 mcM of dexamethasone. After 48 hrs of culture the cells were harvested and the cytokine assays were performed using commercial kits from R&D systems. (Cat #M5000, Cat #M4000B, Cat #M2000, Cat #M 1240).

The result depicted in Table 7 reveals that Mw is effective in suppression of all cytokines like Dexamethasone. The suppressive effect seen is identical to the one observed with 10 mM Dexamethasone. This concentration of Dexamethasone is typically seen as Cmax after administration of 200 mg of Dexamethasone intravenously, 200 mg of Dexamethasone is used in very severe inflammatory conditions as a pulse therapy. Generally it is used at a significantly lower dose as an oral dosage. Generally adults receive 4.0 to 8.0 mg of Dexamethasone per day by oral or parenteral route.

TABLE 7
Cytokine suppression by Mycobacterium w and Dexamethasone
In vitro % inhibition b Mw

| | Mycobacterium w | 10 mM | 10 mcM | 1 mcM |
|---|---|---|---|---|
| IL-5 | 85.1 | 85.81 | 70.3 | 43.9 |
| IL-4 | 95.2 | 93.65 | 845 | 61.9 |
| IL-2 | 78.6 | 70.64 | 33.7 | 25.6 |
| IL-12p40 | 64.4 | 57.69 | 47.1 | 33.7 |

Glucocorticoids like dexamethasone are known anti-inflammatory compounds. The commonly used glucocorticoids include hydrocortisone, prednisolone, betamethasone, dexamethasone, trianiinolone, methytprednisolone, prednisone. They suppress anti-inflammatory as well as proinflammatory cytokines. They are used in management of wide range of diseases which include rheumatoid arthritis, rheumatoid spondylitis, asthma, atopic dermatitis, drug hypersensitivity reactions, perennial or seasonal allergic rhinitis, serum sickness, bullous dermatitis herpetiformis, exfoliative erythroderma, mycosis fungoids, pemphigus, severe erythema multiforme (Stevenes), ulcerative colitis, idiopathic thrombocytopenic purpura, pure red cell aplasia, temporal arthritis, uvetitis, proteinuria in idiopathic nephritis, idiopathic eosinophilic pneumonias, symptomatic sarcoidosis, acute gouty arthritis, ankylosing spondylitis, dermatomyositis, polymyositis, systemic lupus, refractory multiple myeloma, myelodysplastic syndromes, severe COPD, chronic granulomatous disease, angiogenesis, sarcoidosis. Thus all the disease/disease condition including the up-regulation of cytokines, interleukins and chemokines can be treated with Mycobacterium w and/or its constituents with more effective suppression of the said “inflammatory and anti-inflammatory cytokine suppression.

1-19. (canceled)

20. A method for inhibiting p38 protein kinase mediated condition in a subject, comprising administration of an effective amount of Mycobacterium w and/or constituents of Mycobacterium w in said subject.

21. (canceled)

22. The method of claim 20, wherein said inhibition of p38 kinase mediated condition inhibits, suppresses or blocks TNF-α, cytokines, inflammation, cell differentiation, cell proliferation, or cell cycle regulation.

23. The method of claim 20, wherein said inhibition of p38 kinase mediated condition induces apoptosis, anti-inflammatory reactions, immune modulation, vascularization, response to external stimuli or angiogenesis.

24. The method of claim 20, wherein said p38-kinase mediated condition is an abnormality or a disorder in said subject.

25. The method of claim 24, wherein said abnormality or disorder is selected from the group consisting of: arthritis, inflammatory diseases other than asthma, autoimmune diseases, destructive bone disorders, proliferative disorders including tumor progression, infectious diseases, neurodegenerative diseases, allergies, reperfusion, ischemia in stroke, heart attacks, angiogenic disorders, organ hypoxia, vascular hyperplasia cancer cachexia, cardiac hypertrophy, thrombin-induced platelet aggregation, conditions associated with prostaglandin endoperoxidase synthase-2, cancer, immunodeficiency disorders, cell death, and osteoporosis.

26. The method of claim 20, wherein said administering is in vitro or in vivo.

27. The method of claim 20, wherein said subject is a mammal.

28. A method of modulating the activation state of a p38 kinase in a subject comprising the step of contacting a cell expressing said kinase in said subject with Mycobacterium w and or a constituent thereof.
